(19) United States
(12) Patent Application Publication
(10) Pub. No.: US 2009/0274673 A1
(43) Pub. Date: Nov. 5, 2009

(54) COMPOSITION FOR TOPICAL TREATMENT OF ACNE

(76) Inventor: Haig Alexander Eskandarian, New York, NY (US)

Correspondence Address: JAMES C. EAVES JR.;GREENEBAUM DOLL & MCDONALD PLLC, 3500 NATIONAL CITY TOWER, 101 SOUTH FIFTH STREET, LOUISVILLE, KY, 40202, US

(21) Appl. No.: 12/433,987

(22) Filed: May 1, 2009

Related U.S. Application Data
(60) Provisional application No. 61/049,459, filed on May 1, 2008.

(51) Int. Cl. A61K 35/74; A61P 17/10

(52) U.S. Cl. 424/93.46

(57) ABSTRACT

The composition described is to be applied topically to the skin to treat acne and other skin disorders caused in part or wholly by bacterial or mycosal infections. Acne and related skin disorders have complex causes. This formula contains compounds, including an effective amount Bacillus amyloliquefaciens (Bam), that have distinct functions in reducing the sebum on the surface of the skin and inhibiting the proliferation of microbes and fungi where the skin has been wounded or in sebaceous pores. This formula also aims to increase the rate of healing of scars caused by acne by inhibiting opportunistic bacteria from colonizing the wound, while ensuring that healthier skin remains protected from such parasitic bacteria.

### PRIOR APPLICATION

This application claims the benefit of U.S. Provisional Patent Application No. 61/049,459 filed May 1, 2008.

### TECHNICAL FIELD

This invention relates to a topical treatment for acne or other skin disorders.

### BACKGROUND OF THE INVENTION

Acne is a very common disorder that affects millions of adolescents and adults. It is often characterized by the presence of lesions, comedones, papules (pustules), cysts (nodules), post-inflammatory pigmentation, and scarring. Its severity is directly correlated to the quantity and variety of these symptoms. Acnegenesis has been extensively researched and is discussed in U.S. Pat. No. 4,372,296 issued Feb. 8, 1983, to Mostafa S. Fahim. However, identifying the causes of acne is as much an art as a science. The complexity of the disorder may be better understood by integrating all the additive causes of acne into a comprehensive network. Connections between the various causes and the host epidermis represent the interactions among the causes and between the causes and the host. To ensure that the onset of acne is thoroughly understood, it is necessary to rectify variations in the interactions between the microflorum and many host patients. The normalization of observations and diagnoses from patient to patient can lead to the creation of classification tables or complex algorithms to model acne's genesis.

Acne typically begins at puberty, when an increase in the expression of androgens increases the size of the pilosebaceous glands. The larger unit, comprised of the hair shaft, hair follicle, sebaceous gland, and erector pili muscle, allows hair to grow and, more importantly, allows the sebaceous gland to secrete more sebum. The sebum, comprised of lipid-based film, functions as a protective barrier for the skin and inhibits the drying of the skin, while also acting as a medium upon which opportunistic bacteria can grow. The localization of acne is directly related to the distribution of sebaceous follicles on the face, neck, and back. Because sebum is a highly lipid-rich medium, it acts as an effective barrier for inhibiting water loss through the epidermis. However, this also allows water-insoluble substances to accumulate and even enter follicular shunts, allowing bacteria to bypass the horny layer of the skin, which also confers water resistance. An example of the high uptake of water-insoluble chemicals is the passage of corticosteroids through the huge canals of sebaceous follicles. This formula will aim to reduce the level of sebum that is secreted. However, overuse of the formula could, as with over-washing with soaps, cause the skin to become excessively dry due to the loss of the lipid-soluble barrier. In light of this, the concentrations of compounds associated with decreased sebum levels on the epithelium are to be closely controlled.

The microflora attributed with the onset of acne have been studied at length. The bacteria and fungi inhabiting the skin are characterized by the presence of C. acnes (in the canals of sebaceous follicles), C. cocci (in Baird-Parker type II follicular mouths), and the yeast-like fungi P. ovale and P. orbiculare (also inhabiting predominantly follicular mouths)₃. A very common strain inhabiting all known humans is Staphylococcus epidermidis, the most prominent coagulase-negative staphylococcal species colonizing human skin and mucous membranes. S. epidermidis (Sep) has two phases of colonization of various tissues other than the skin and mucous membranes: the primary attachment and the bacterial accumulation phases. Hydrophobic interactions contribute to the initial accumulation of Sep. The staphylococcal surface protein Ssp1, capsular polysaccharide adhesin (PS/A), autolysin AtlE, and fibrinogen binding protein (Fbe) all contribute to the attachment of Sep to the host surface. Fibronectin and fibrinogen produced by the human host attract bacterial colonization by Sep. Bacterial accumulation ensues when polysaccharide intercellular adhesin (PIA) mediates intercellular adhesion, and when hemagglutinin and accumulation associated protein (AAP) are expressed 5.

Microbial and mycosal infections are typically treated with common antibiotics like erythromycin or tetracycline. However, several strains of bacteria have evolved antibiotic resistance against these various compounds. Thus, it is beneficial to discover novel antimicrobials and antimycosals to which antibiotic resistance has not yet developed in the epidermal microflora in order to inhibit and eliminate bacterial and fungal growth. The antibiotics of the present invention are active only against their respective microorganisms and do not have the capability to penetrate or harm human cells. Furthermore, antimicrobials used at concentrations significantly below the toxicity levels (IC₅₀) for humans are beneficial in treating a disorder such as acne.

Iturin A is a fungicide that has been proposed as an antimycosal agent to increase the longevity of stored grain. This cyclic lipopeptide, produced by the spore-forming, gram-positive bacterium Bacillus subtilis (Bsu), has been tested on plants and humans in the hope it would prove beneficial in treating both hosts. Bsu has been known to produce various antibiotics like bacillomycin and mycosubtilin that have antimycosal activities. The antimycosal agent inhibits the growth of species of Fusarium, Gerlacia, Penicillium, and Aspergillus. Bsu is a versatile bacterium that has been studied in great detail by developmental geneticists interested in understanding how it develops into a metabolically dormant spore that can survive high environmental stresses and nutrient starvation for centuries. However, it is only one of several spore-forming bacteria that are being studied increasingly for their applied uses in very diverse areas of human society.

Another species of the spore-forming Bacillus family, Bacillus amyloliquefaciens (Bam), has been cultured en masse to isolate the protein α-amylase for use in the dry-cleaning industry. Bam has very important agricultural benefits, as it can encourage root growth. It is referred to as a plant growth-promoting rhizobacterium. Bam competitively colonizes plant roots and acts as an antagonist for plant-borne pathogens. It is now known that over 7% of the Bam genome is dedicated to the biosynthesis of polyketides and peptides, enabling the bacterium to effectively compete with other organisms within the plant rhizosphere. It does this by competing for nutrients such as iron and by producing antibiotics and lytic enzymes. Bam FZB42 contains numerous gene clusters encoding cyclic lipopeptides and polyketides (synthesized without action by the ribosome) with distinct antibacterial functions. Iturin lipopeptides are antimycosal compounds. The most important ones include iturins like Iturin A, mycosubtilin, and bacyllomycin D, and surfactin and fengycins. Iturins like mycosubtilin and Iturin A, studied nearly two decades ago, were postulated to function in the following mechanism: the iturin molecules penetrate the cytoplasmic membrane of the target mycosal, nematode, or bacterial cell. They then disorganize the lipid bilayer by activating phospholipases, form inside the membrane oligomeric ion-conducting structures, allowing the leakage of intracellular K⁺, and finally permeabilize the cell membrane of fungi while interacting with phospholipids and forming complexes with sterols.

The antibiotic activity of Bam is attributed in part to its expression of polyketides. One known polyketide that has been studied recently and has shown promise as an antibacterial product is bacillaene. The synthesis of polyketides is controlled by three large modular PKS systems in Bam strain FZB42. Furthermore, the gene clusters pks1 (bae) and pks3 (dij) encode the biosynthesis of the polyene antibiotics bacillaene and difficidin. Bacillaene was discovered in Bacillus subtilis 3610 to inhibit the growth of Streptomyces avermitilis by conducting gene knockout studies of the pksX locus where bacillaene biosynthesis genes are encoded. Bacillaene has been found to inhibit prokaryotic life at the level of protein synthesis. Furthermore, it is selective, inhibiting protein synthesis only in prokaryotes and not in eukaryotes. In studies published alongside the biochemical results just stated, bacillaene was determined to be ineffective against Saccharomyces cerevisiae or Candida albicans (a yeast-like fungus) and, interestingly, Staphylococcus epidermidis. However, its inhibitory effect, as quantified by agar plate diffusion assays, was observed to be potent against hyper-permeable strains of Escherichia coli SC10909 and BAS847 and moderately potent against Proteus vulgaris, Bacillus thuringiensis, and Staphylococcus aureus. The inhibition of Streptomyces family organisms is indicative of this antibacterial's potential robustness against the growth not only of gram-positive bacteria (B. thuringiensis) but also against diverse groups of gram-negative (E. coli) bacteria.

### SUMMARY OF THE INVENTION

The present invention provides a topically-applied compound which includes an effective amount of Bacillus amyloliquefaciens (Bam).

### DETAILED DISCLOSURE

This anti-acne cream incorporates an antimycosal iturin and an antibacterial polyketide into a topical application that aims also to reduce sebum on the skin surface. Three major active components of the cream will sequentially function as a cleanser to wash away the medium in which bacteria and fungi live and through which they enter the sebaceous pores of the epidermis and cause an acnetic outbreak.

The formulae disclosed in this application is to be administered topically to treat acne and other skin disorders caused fully or in part by opportunistic bacteria and fungi that invade and colonize the epidermis.

The formula for the acne cream is presented here in a form that aims to treat moderate or chronic acne. However, it is also possible to dilute the formula or modify one chemical compound to make the cream less potent and therefore more appropriate for patients suffering from acute or mild forms of acne.

Zinc salt has been studied in great detail with regards to its importance in biological systems. It is a limiting factor in the formation of RNA and DNA as well as polymerases, kinases, and several other enzymes. Zinc is well known in protein biochemistry for its structural role in the creation of zinc fingers by binding histidine and cysteine residues and cross-linking them together to form a loop structure. The concentration of zinc ions is also an indication of whether cells will undergo apoptosis when starved of Zn. A recent study showed that exposure to high concentrations of zinc could disrupt iron homeostasis in yeast while also inducing oxidative stress response genes that function in iron and sulphur regulation and metabolism. Thus, its concentration in the cell is regulated and its addition to the skin could act to keep healthy skin that would otherwise be menaced by pathogenesis. Zinc pyrithione salt has been quantified within different parts of the human body, and skin tissue has been observed to have a higher concentration of this salt. Studies have suggested that zinc acts as an antimycoside against Malassezia furfur. Studies have also shown that zinc oxides can be used to treat psoriasis and dandruff by inhibiting the over proliferation of cells. Zinc chloride has been used to reduce the likelihood of sunburn by UV irradiation. The salt has also been observed to decrease viral load. Tests have even shown that HIV infectivity was reduced when the virus was incubated with zinc acetate. The stimulation of epidermal basal cells of mice and the increased expression of insulin-like growth factor 1 and other mRNAs are attributed with healing wounds, and zinc has been shown to induce such activities. This cream aims to incorporate 0.5% zinc oxide (ZnO) by weight in order to induce healing of skin wounded by acne.

The addition of an amino acid like L-Glycine or D,L-Lysine allows zinc oxide to react with the monocarboxylic acid of glycine while also conferring water solubility on the zinc ions. Thus, L-Glycine will be used to supplement zinc sulfate in a concentration of 10 molar equivalents to zinc sulfate. The zinc-glycine complex will form as Zn(C₂H₄—NO₂)₂.2H₂O using an anhydrous form of glycine. A recent study suggests that adding nutrients to re-stimulate bacterial growth after a population has reached a static growth phase could briefly allow for a window of vulnerability to antibiotics of the re-induced bacteria. Amino acids act as nutrient sources to push bacteria back into a growth phase. Unfortunately, dicarboxylic acids, Aspartate or Glutamate, amino acids often used to stimulate bacterial growth, are poor candidates for incorporation into this cream, which also rules out the possibility of using these residues to make microorganisms more susceptible to killing by antibiotics.

Ascorbic acid (3% by weight) will supplement the formula in order to decrease the amount of sebum on the surface of the epidermis. This will confer the added benefit of reducing the medium through which microorganisms travel to enter sebaceous pores of the host epidermis. To prevent the oxidation of ascorbic acid and increase the length of storage time, the cream will be supplemented with vitamin E-DL-α-tocopherol (100 IU/g). Vitamin A (1000 IU/g) will be added to the cream to smooth the skin.

To ensure that it functions as an antimycosal and antibacterial cream, bacillaene, a polyketide, will be added at a concentration of 5.5 μg/ml. This concentration will ensure that a lethal dose will be administered to most colonizing bacteria. The antimycosal product Iturin A will supplement the cream at a concentration of between 10 and 25 μg/ml. Together, the iturin lipopeptide and polyketide will reduce the surviving microflora on the epidermal surface, and ascorbic acid and zinc salt will reduce the level of sebum film on the surface of the skin.

To make it possible to administer the supplements mentioned above topically, the mixture can be dissolved in purified water aliquoted with 4% methyl cellulose into 50.0 mL, a gel-like substance equivalent to Vehicle N (sold by the Neutrogena Corporation), which is composed of ethyl alcohol, isopropyl alcohol, propylene glycol, and water. Another carrier base that can be used by patients with skin sensitive to alcohol-based creams is the following: 48.0 g lubricating jelly, 1.1 g methyl parabens, 0.3 g propyl parabens, and 5.0 ml propylene glycol. The base mixture can be greatly varied to add pleasant odors and colors to the cream. However, it is important to ensure that zinc chelators such as EDTA, citric acid, tartaric acid, orthophenanthroline, and others are not added. The carrier base may contain such compounds as chamomile extract, calendula extract, fragrance oils, jasmine extract, vaccinium corybosum, coffea Arabica extract, carica papaya extract, malus domestica extract, methyl/propylparaben, etc. The carrier base may also be made more viscous by supplementing it with cetyl alcohol, stearyl alcohol, white petrolatum, mineral oil, or propylene glycol. It may also be produced as a less viscous substance by increasing the volume of water in relation to the other supplements. The carrier base must be adjusted to a pH of between 6 and 7, preferably near a pH of 6.5, to ensure the antibiotics remain properly folded.

The acne cream will be applied topically on a daily basis while the face and neck region are being washed. The antibacterial and antimycosal supplements will reduce the amount of colonizing opportunistic bacteria and fungi on the treated skin while the sebum level will be reduced by ascorbic acid and zinc sulfate polyhydrate. The aim of the drug is to decrease the severity of acne, but it is not confined to this function. It could potentially act to decrease viral load that could by chance temporarily find itself on the host epidermis. Though antibiotics have been observed to reduce HIV-1 infectivity and even herpes lesions, it is not likely that these viruses will be present in the organ system this product aims to treat; thus, these uses might be considered irrelevant with regards to the aim of the proposed topical cream. Nonetheless, the drug's potential effect on reducing viruses on the epidermis cannot be completely ignored, as it could serve some novel benefit in the future. Still, the formula explicated in this disclosure is specifically designed for use on facial acne and acne on the neck and back.

The foregoing description of a preferred embodiment of the invention has been presented for purposes of illustration and description. It is not intended to be exhaustive or to limit the invention to the precise form disclosed. Other modifications or variations are possible in light of the above teachings. The embodiment was chosen and described in order to best illustrate the principles of the invention and its practical application to thereby enable one of ordinary and skill in the art to best utilize the invention in various embodiments and with various modifications as are suited to the particular use contemplated. It is intended that the scope of the invention be defined by the claims appended hereto.

What I claim is:

1. A composition for topical skin application for the treatment and prevention of acne and other skin disorders comprising:
an effective amount of Bacillus amyloliquefaciens (Bam); and
a pharmaceutically acceptable carrier base.
